(19) United States
(12) Patent Application Publication
(10) Pub. No.: US 2010/0216891 A1
(43) Pub. Date: Aug. 26, 2010

(54) AROMATIC ANTIHYPERTENSIVE AGENT, AND METHOD FOR LOWERING BLOOD PRESSURE IN MAMMALS

(75) Inventors: Ken SHIMONO, Osaka (JP); Hiroaki Oka, Osaka (JP); Masato Suzuki, Osaka (JP); Kanae Senda, Nara (JP); Shoji Komai, Nara (JP)

Correspondence Address: McDERMOTT WILL & EMERY LLP, 600 13th Street, N.W., Washington, DC, 20005-3096, US

(73) Assignee: PANASONIC CORPORATION, Osaka (JP)

(21) Appl. No.: 12/773,534

(22) Filed: May 4, 2010

Related U.S. Application Data
(63) Continuation of application No. PCT/JP2009/001039, filed on Mar. 9, 2009.

(30) Foreign Application Priority Data
Mar. 13, 2008 (JP) 2008-063658
Oct. 1, 2008 (JP) 2008-256028

(51) Int. Cl. A61K 31/121; A61P 9/12

(52) U.S. Cl. 514/675

(57) ABSTRACT

The present invention provides an antihypertensive agent having a superior antihypertensive action, and a method for lowering blood pressure in mammals by inhalation of such an antihypertensive agent through the nostril.

The antihypertensive agent of the present invention comprises citral and linalool, and has a mass ratio of citral to linalool of not less than 10:1 and not greater than 40:1. Since this antihypertensive agent has a superior antihypertensive action, it can be utilized as antihypertensive compositions for medical drugs, foods and beverages and feeds.

This is a continuation application under U.S.C 111(a) of pending prior International application No.PCT/JP2009/001039, filed on Mar. 9, 2009, which in turn claims the benefit of Japanese Application No. 2008-063658 filed on Mar. 13, 2008 and Japanese Application No. 2008-256028 filed on Oct. 1, 2008, the disclosures of which Application are incorporated by reference herein.

### BACKGROUND OF THE INVENTION

1. Field of the Invention

The present invention relates to an antihypertensive agent, and more specifically relates to an antihypertensive agent containing particular aromatic components included in plants and the like. In addition, the present invention relates to a method for lowering blood pressure in mammals by nasal inhalation of such an antihypertensive agent.

2. Related Art

Conventionally, it has been empirically known that aromas have a variety of psychological and physiological effects, and aromatherapies using aromatic oils (essential oil) etc., extracted from plants have been prevalent for stabilizing mood, relieving stress, as well as for targeting insomnia.

In an exemplary attempt to demonstrate physiological effects produced by an aromatic composition used in aromatherapies, it has been reported that an aromatic composition containing linalool as an active ingredient causes a variety of physiological changes involving the blood pressure and body temperature regulation in anesthetized rats (Patent Document 1).

It has been also reported that particular aromatic components included in plants have an antihypertensive action (Patent Document 2) or an improving action of brain functions (Patent Document 3).

However, effects produced by aromas of plant essential oils on treatments of diseases have been still uncertain, and just only a few functions of each of the components have been found.

### PRIOR ART DOCUMENT

Patent Document 1: Japanese Patent Laid-open Publication No. 2005-206596

Patent Document 2: Japanese Patent Application Publication No. Hei 3-019210

Patent Document 3: Japanese Patent Laid-open Publication No. 2007-302572 (particularly, paragraph No. 0016)

Patent Document 4: Japanese Patent Laid-open Publication No. 2005-206597 (particularly, paragraph No. 0008)

Patent Document 5: Japanese Patent Laid-open Publication No. Hei 3-277368 (particularly, FIG. 1, FIG. 4, FIG. 7 and FIG. 8, and detailed description of these)

Patent Document 6: Japanese Utility model Publication No. Hei 5-4840

### SUMMARY OF THE INVENTION

Some of plant essential oils which have been used in aromatherapies produce an antihypertensive action as suggested in prior arts. However, essential oils purified from plants include various components, and further the ratio of the components may vary depending on the production district. Therefore, it has been still unclear as to which component has an antihypertensive action in effect.

The present invention solves the foregoing problems in prior arts, and an object of the invention is to provide an antihypertensive agent having a stable effect irrespective of its product district or purification method, by identifying a component having an efficient antihypertensive action.

The antihypertensive agent according to the present invention that solves the problems of the foregoing prior art contains citral and linalool, and has a mass ratio of citral to linalool being not less than 10:1 and not greater than 40:1. It is desired that the mass ratio of citral to linalool is 20:1.

The above objects other objects, features and advantages of the present invention will be apparent from the following detailed description of preferred embodiments with reference to attached drawings.

According to the present invention, an antihypertensive agent having a superior antihypertensive action is provided. Additionally, according to the present invention, by inhalation of such an antihypertensive agent through the nostril, the blood pressure in mammals is lowered. It is to be noted that human blood pressure is also lowered in the method of the present invention, and the blood pressure in nonhuman mammals is also lowered.

### BRIEF DESCRIPTION OF THE DRAWINGS

FIG. 1 shows a graph illustrating the change in diastolic blood pressure observed when various aromatic compositions are presented to rats;

FIG. 2 shows a graph illustrating the change in diastolic blood pressure of rat observed when the ratio of blending in the aromatic composition is altered;

FIG. 3 shows a perspective view illustrating a blood pressure measurement device which can be utilized as one exemplary electronic device for inhalation of the antihypertensive agent of the present invention through the nostril;

FIG. 4 shows a cross-sectional view partially enlarged from FIG. 3;

FIG. 5 shows a control block diagram of FIG. 3;

FIG. 6 shows a configuration diagram illustrating a sauna apparatus which can be utilized as one exemplary electronic device for inhalation of the antihypertensive agent of the present invention through the nostril;

FIG. 7 shows a control block diagram of FIG. 6;

FIG. 8 shows a configuration diagram illustrating a toilet apparatus which can be utilized as one exemplary electronic device for inhalation of the antihypertensive agent of the present invention through the nostril;

FIG. 9 shows a cross-sectional view partially enlarged from FIG. 8; and

FIG. 10 shows a control block diagram of FIG. 8.

### DETAILED DESCRIPTION OF THE INVENTION

Hereinafter, one embodiment of an antihypertensive agent that embodies the present invention will be explained in detail.

The antihypertensive agent of this embodiment is an antihypertensive agent characterized by containing citral and linalool, and having a mass ratio of citral to linalool being no less than 10:1 and no greater than 40:1.

Hypertension promotes lesion of heart and blood vessel, and has been considered as a risk factor to which the greatest importance should be attached in cardiac diseases and cerebral apoplexy. As methods for treating hypertension, nonpharmacological therapies such as alimentary therapies, and appropriate exercise, as well as pharmacological therapies using a β blocking agent or a calcium antagonist have been carried out. However, hypertension cannot be ameliorated with dietary restriction or an exercise therapy in many cases, and the effects of antihypertensive agents may not be equally achieved depending on the cases. Moreover, since a hypertension treatment may be continued for a long period of time, a safe therapy with no fear of side effects has been desired.

Blood pressure is controlled by four processes in rough aspects, i.e., regulation by a cardiac contracting function, regulation of the body fluid volume by kidney, regulation of vasoconstriction states by an autonomic nerve system, and regulation of vasoconstriction states by humoral factors such as hormones. Regulation by nerves starts from perception of blood pressure elevation at baroreceptors present in carotid sinus and aortic arch. Excitation of baroreceptors is transferred via the vagal nerve and glossopharyngeal nerve to the vasomotor center and cardioinhibitory center. Then, sympathetic tone is decreased, and thus vagal tone is increased concomitant with dilation of the blood vessels, leading to decrease in the heart rate, and lowering of the blood pressure. Upon blood pressure lowering, a reverse reaction occurs.

On the other hand, it has been known that cerebral limbic system that reportedly participates in the autonomic nervous system is strongly reflected from the olfactory nerve system that receives an odorant. In other words, some odorants are believed to affect a variety of physiological actions via the olfactory nerve system and the cerebral limbic system.

Since the antihypertensive agent of this embodiment is an aromatic composition containing citral and linalool, it is active in effectively lowering the blood pressure via the olfactory nerve system, the cerebral limbic system, and the autonomic nervous system. In addition, the aromatic composition characterized by having the upper limit of a proportion of linalool to citral of one-tenth, and the lower limit thereof of one-fortieth has a particularly efficacious antihypertensive action. It is considered that such an antihypertensive phenomenon is caused by suppression of a sympathetic nerve that controls kidney and suppression of a sympathetic nerve that controls the adrenal gland which cooperate in combination. In this respect, the antihypertensive agent of the present invention can be suitably used as a composition for lowering the blood pressure, or as a composition for ameliorating a hypertensive state.

The antihypertensive composition of the present invention can be blended as a flavor (aromatic component) having an antihypertensive action into a variety of products, for the purpose of imparting the function (efficacy) to an object product. Accordingly, the present invention provides an antihypertensive composition comprising the antihypertensive agent of the present invention.

In the present invention, an antihypertensive composition means a product comprising the aforementioned antihypertensive agent of the present invention, and consequently develops an odor (aroma) resulting from citral and linalool. The antihypertensive composition intended by the present invention has an action of lowering the blood pressure based on the development of the odor resulting from the citral and linalool described above. The antihypertensive composition of the present invention is not limited as long as it has an antihypertensive action, and is preferably used in applications utilizing its action (for lowering blood pressure). Specific examples of the antihypertensive composition include e.g., aromatic substances, perfuming cosmetic products, inhalants, detergents for textile products, textile products, building material, foods and electrical appliances.

These antihypertensive compositions may also be a product to which an indication for showing a particular function (efficacy) or application is attached to meet their antihypertensive action and application on the basis that the antihypertensive agent of the present invention is contained.

Examples of the aromatic substances include aromatic substances having an action of lowering the blood pressure by containing the antihypertensive agent of the present invention, and thus used for lowering the blood pressure. As long as these features are satisfied, any aromatic substances such as aromatic substances for room, aromatic substances for toilet, aromatic substances for automobile, aromatic substances for entrance hall, aromatic substances for bathroom, aromatic substances for closet, aromatic substances for fibers such as clothes, aromatic substances for chest are acceptable.

In addition, aromatic substances referred to herein also include products having a deodorant effect in combination. The form of the aromatic substance may be selected from various types of forms such as solid forms, semi-solid forms (jelly state or gel state), liquids and aerosols.

Examples of the perfuming cosmetic products include perfuming cosmetic products having an action of lowering the blood pressure by containing the antihypertensive agent of the present invention, and thus used for lowering the blood pressure. These perfuming cosmetic products may include cosmetics, washing agents such as soaps, fragrances such as bath agents and perfumes, toothpastes, liquid dentifrices, mouth washes, mouse pets and mouth fresheners. Specific examples of the perfuming cosmetic products include e.g., creams, milky lotions, cosmetic powders, body lotions, hair dressings, hair washes, soaps, body soaps, antiperspirants, shampoos, rinse agents, perfumes, skin lotions, lip sticks, lip balms, sun screens, massage oils, massage creams and skin care oils.

Examples of the inhalants include such forms having an action of lowering the blood pressure by containing the antihypertensive agent of the present invention, and thus used for lowering the blood pressure. Although specific forms of these inhalants are not limited, they may be in the form that permits an aroma to be generated in the vicinity of the nostril by spraying or volatilizing of the aromatic component, thereby allowing the aroma to be intentionally inhaled, and for example, nasal drops, air sprays and inhalable liquids are exemplified.

The foods may be those having an action of lowering the blood pressure by containing the antihypertensive agent of the present invention, and thus used for lowering the blood pressure. In order to achieve the effects by the aromatic components (citral and linalool) of the antihypertensive agent of the present invention efficiently, foods that stay in the mouth for a comparatively long time and allow the aroma to be inhaled via the nasal cavity, as well as foods that allow the aroma components to be inhaled through the nasal cavity before thrown into the mouth are preferred. Specific examples include confectioneries such as gums, candies, gummy candies and troches; desserts and frozen desserts such as ice creams and yogurt; refreshing beverages such as tea; mouth refrigerants; and supplements.

Examples of the detergents for textile products include detergents having an action of lowering the blood pressure by containing the antihypertensive agent of the present invention, and may include anionic, cationic, nonionic or amphoteric ionic detergents, textile product softening agents, products for softening textile products, and fiber softening agent products for use in dryers.

Examples of the textile products include those having an action of lowering the blood pressure by containing the antihypertensive agent of the present invention, and may include clothes such as underwear (also including socks, tights and stockings) and outer wears, handkerchiefs and towels, sanitary goods such as masks and sweat absorbing pads, rugs, bags and pouches, curtains, beddings (top covers, sleeping mats, rug mats, blankets, cotton blankets, pillows, bed mats, sofas, cushions, cushions for sitting, and covers of these) and fancy goods.

Examples of the building material include materials having an action of lowering the blood pressure by containing the antihypertensive agent of the present invention, and thus used for lowering the blood pressure, which are preferably exemplified by building materials used for lowering the blood pressure on the basis of such an action. Although specific forms of these are not limited, they may be in the form of wallpapers, various guardian gods, fusuma papers, boards and paint materials.

Examples of the electrical appliances include those having an action of lowering the blood pressure by containing the antihypertensive agent of the present invention, and electrical appliances used for lowering the blood pressure on the basis of such an action may be exemplified. Although specific forms of these are not limited, they may be in the form of air conditioners or air cleaners.

Examples of the quasi drugs include those having an action of lowering the blood pressure by containing the antihypertensive agent of the present invention, and thus used for lowering the blood pressure on the basis of such an action. Although specific forms of these are not limited, they may be in the form of toothpastes, liquid dentifrices, mouth washes, mouse pets, mouth fresheners and cataplasms.

The antihypertensive composition of the present invention described in the foregoing includes a composition characterized by having the upper limit of a proportion of linalool to citral of one-tenth, and the lower limit thereof of one-fortieth contained as aromatic components thereof. Therefore, according to the antihypertensive composition of the present invention, the aromatic components emitted therefrom stimulate the olfactory sense, and can exhibit an antihypertensive action that results from suppression of sympathetic nerve activity or promotion of vagal nerve activity. In other words, the antihypertensive composition of the present invention can be suitably used in applications for lowering the blood pressure, as well as in applications for ameliorating the hypertensive state.

### EXAMPLES

Next, the embodiments will be explained in more detail by way of each Test Example.

### Test Example 1

### Evaluation of Action of Aromatic Composition on Change in Blood Pressure

Focusing attention on aromatic components contained in a Melissa essential oil, one of plant essential oils, various aromatic compositions were presented to male Wister strain rats, and evaluation of change in caudal arterial pressure before and after the presentation was conducted. The test was carried out under conditions that avoid stress which may be placed on the rat as less as possible, and thus can be kept calm in an environment of the measurement of the blood pressure.

<Test Animal>

Male Wister strain rats of 7-week old were purchased, and after one week acclimatization period, the blood pressure was measured on 8-week old. All experimental animals were kept under a 12:12 light-dark cycle (light period: from 8 AM to 8 PM), with allowing for free access to chow and drinking water.

<Training for Adaptation to Test>

Training for adaptation of the 7-week old rat to a holder for the blood pressure measurement was carried out for one week. The training was started from one week before the measurement date, and a time period kept in the holder was prolonged daily, and the air passed through a mineral oil was presented to the vicinity of the rat nostril from three days before the measurement date. Furthermore, from two days before the measurement date, a cuff for measuring the blood pressure was attached to the rat tail, and thus adaptation to the pressure applied during the measurement was also permitted. All the trainings were performed during the light period, and from two days before the measurement date, the training was performed at just around the same time as each measurement time.

<Blood Pressure Measuring Method>

For three days after completing the training, change in the blood pressure was measured upon presentation of the aromatic composition to the rats. The measurement schedule was made so as to eliminate the influences of the circadian rhythm to as less as possible. For the measurement of the blood pressure, an unheated non-invasive blood pressure manometer for mice and rats (Muromachi Kikai Co., Ltd., Model MK-2000ST) was used, and the rat was kept in the provided holder, having the cuff for measuring the blood pressure attached on the tail root. The rat was kept in the holder from ten minutes before the measurement, and after visually checking the rat to be calm based on the movement of the head and tail, the measurement of the blood pressure was started. The blood pressures before and after 30 minutes from presenting the aromatic compositions were measured, and the changes of the blood pressures were compared. The measurement data at each time point were represented by average values of the diastolic blood pressure from five times measurements at each time points, respectively. Values decided to be abnormal were eliminated on the basis of the displayed waveform on the blood pressure manometer, or observation of body motion of the rat.

<Method of Presenting Aromatic Composition>

During the measurement of the blood pressure, the air which had been passed through activated charcoal, silica gel and mineral oil (flow rate: 50 cc/min) was constantly kept supplying to the vicinity of the nostril of the rat. When the aromatic composition was presented, the air which had been passed through a diluted liquid with the mineral oil beforehand was presented to the vicinity of the nostril of the rat for 10 minutes. The presented aromatic composition was selected so as to be fresh for each of the rats.

<Presented Aromatic Composition>

The aromatic compositions used in this test were: Melissa essential oil, citral (manufactured by Wako Pure Chemical Industries, Ltd.; specific gravity 0.89 g/ml) and linalool (manufactured by Aldrich Corp.; specific gravity 0.86 g/ml) which are components contained in the Melissa essential oil, and a mixture of citral and linalool. Melissa, citral, and linalool employed were diluted preparations, each of 0.5 μL with 500 μL of the mineral oil (manufactured by Nacalai Tesque, Inc.; specific gravity: 0.87 g/ml). The mixture of citral and linalool employed was a solution prepared by dissolving 2 μL of citral and 0.1 μL of linalool in 502.9 μL of the mineral oil (mass ratio of citral to linalool: about 20:1).

<Test Results>

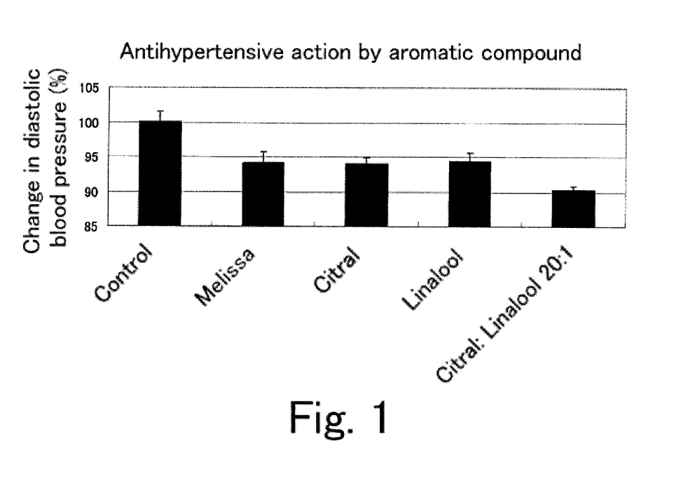

As shown in FIG. 1, lowering of the blood pressure was found out with all the aromatic compositions used in this test at 30 minutes after the presentation as compared with the values before the presentation. Particularly, the composition in which citral and linalool are mixed at a ratio of 20:1 exhibited a remarkable difference (critical rate: not greater than 1%) as compared with the control case in which the air including only the mineral oil was presented.

### Test Example 2

### Evaluation on Antihypertensive Action Depending on Blend Ratio of Citral and Linalool

For the mixture of citral and linalool which demonstrated in Test Example 1, evaluation of the antihypertensive action depending on the blend ratio was carried out. The method of measuring the change in the blood pressure in this Test was similar to the method in Test Example 1.

<Presented Aromatic Composition>

The aromatic compositions used in this Test were five kinds of mixtures having amass ratio of citral to linalool of 4:1, 10:1, 20:1, 40:1 and 100:1, respectively. Specifically,

(1) a solution of 2 μL of citral and 0.5 μL of linalool dissolved in 502.5 μL of mineral oil (mass ratio of citral to linalool, about 4:1);

(2) a solution of 2 μL of citral and 0.2 μL of linalool dissolved in 502.8 μL of mineral oil (mass ratio of citral to linalool, about 10:1);

(3) a solution of 2 μL of citral and 0.1 μL of linalool dissolved in 502.9 μL of mineral oil (mass ratio of citral to linalool, about 20:1);

(4) a solution of 2 μL of citral and 0.05 μL of linalool dissolved in 503 μL of mineral oil (mass ratio of citral to linalool, about 40:1); and

(5) a solution of 2 μL of citral and 0.02 μL of linalool dissolved in 503 μL of mineral oil (mass ratio of citral to linalool, about 100:1) were used.

<Test Results>

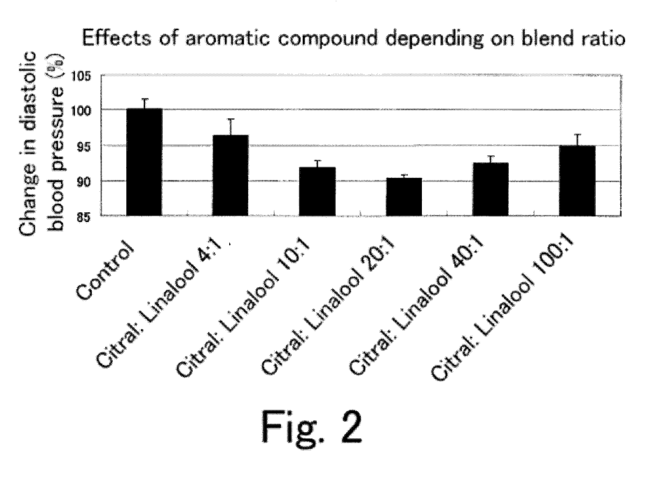

As shown in FIG. 2, also in the examples of blend employed in this test, lowering of the blood pressure was found out at 30 minutes after the presentation as compared with the values before the presentation. With the composition of the mixture having a mass ratio of citral to linalool of 4:1 or 100:1, just a similar level of the effect to the cases of Melissa, citral and linalool that revealed lowering of the blood pressure in the results of the Test 1 described above was found. On the other hand, with the composition of the mixture having a mass ratio of citral to linalool of 10:1, 20:1 or 40:1, a significant difference was ascertained as compared with the cases of Melissa, citral and linalool that revealed lowering of the blood pressure in the results of the Test 1 described above was found. Of these, the greatest antihypertensive action was found out when the mass ratio was 20:1.

The antihypertensive agent of the present invention can lower the blood pressure in a mammal by allowing it to be inhaled through the nostril of the mammal by using the electronic device as described below.

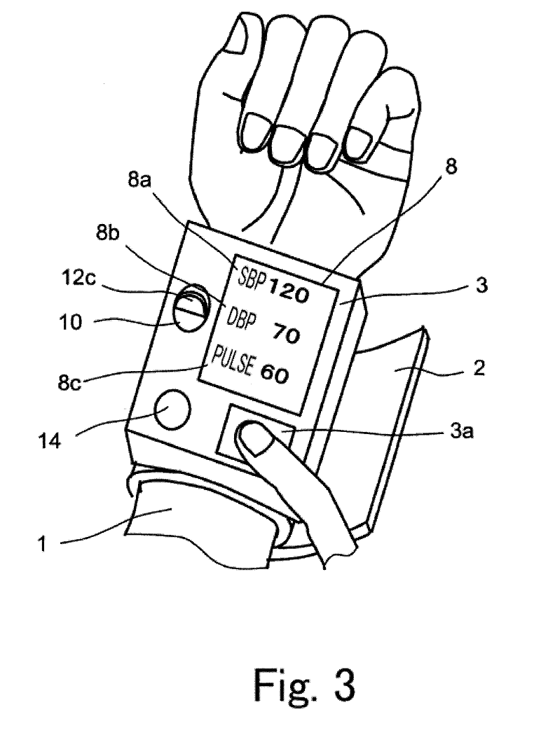

FIG. 3 to FIG. 5 show a blood pressure measurement device which can be utilized as one exemplary electronic device for inhalation of the antihypertensive agent of the present invention through the nostril of a mammal.

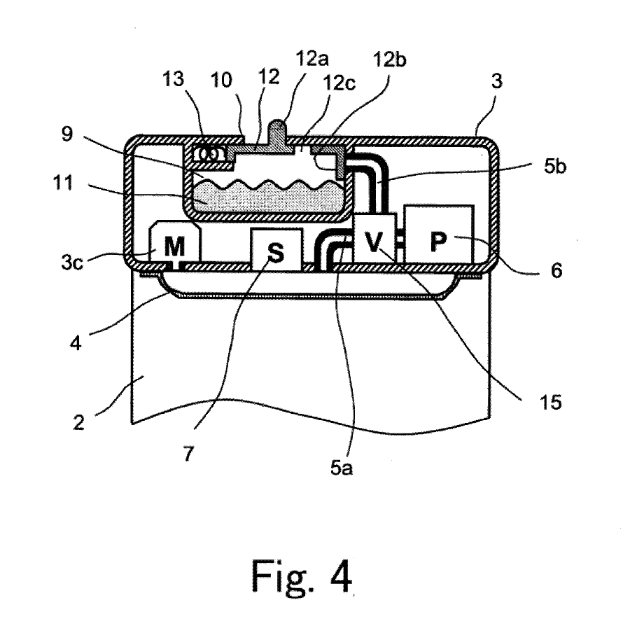

The present blood pressure measurement device has, as shown in FIG. 3 and FIG. 4, a harness (cuff) 2 attached to the arm 1 of a human body, a main body case 3 having a substantially flat box-like shape equipped to this harness 2, a pressurization bag 4 provided at least one of the main body case 3 or the harness 2, a blower (air blasting pump) 6 for compressing the air into the pressurization bag 4 through a draft air duct 5a, and a blood pressure measurement unit (pressure sensor) 7 for measuring the blood pressure in the human body after compression to the arm 1 by the pressurization bag 4.

At the center of the upper surface of the main body case 3 is provided a display unit 8 as shown in FIG. 3, for displaying the measurement values by the blood pressure measurement unit (pressure sensor) 7.

In this display unit 8: the upper column 8a displays the systolic blood pressure; the middle column 8b displays the diastolic blood pressure; and further the bottom column 8c displays the pulse.

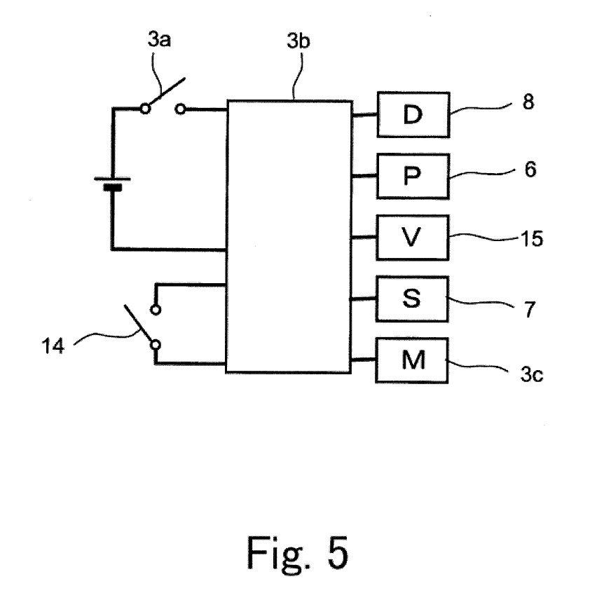

In other words, also the present blood pressure measurement device, the harness (i.e., cuff) 2 is attached to the arm 1 upon the measurement of the blood pressure in a similar manner to preexisting blood pressure measurement devices, and a switch 3a in FIG. 3 and FIG. 5 is manipulated to close in this state.

Thus, a control element 3b drives a blower (air blasting pump) 6 and the air from the blower (air blasting pump) 6 is compressed into the pressurization bag 4 through the draft air duct 5. In the following procedure, the systolic blood pressure and the diastolic blood pressure values, and the pulse can be detected while confirming with Korotkoff sounds with a microphone 3c.

As comprehension of basic configuration and operation of the blood pressure measurement device would have been made from the foregoing description, features of the present blood pressure measurement device are explained below.

In the present blood pressure measurement device, the main body case 3 is provided with an antihypertensive agent storage unit 9 in the form of a vessel, and an opening 10 for allowing the aromatic antihypertensive agent components from the aromatic antihypertensive agent stored in this antihypertensive agent storage unit 9 to flow outside the main body case 3, as shown in FIG. 4.

Furthermore, the lower side in the antihypertensive agent storage unit 9 stores a carrier 11 such as sponge or cotton impregnated with a liquidified aromatic antihypertensive agent.

Moreover, the upper opening 10 is covered with an openable and closable lid 12. This lid 12 is provided with an upper center pinch 12a for enabling movable operation of this lid 12 from the outside of the main body case 3 toward the left side in FIG. 2, a pressure plate 12b at the right side end for enabling moving this lid 12 toward the left side in FIG. 2 upon receiving an air blasting pressure from a blower (air blasting pump) 6 in the main body case 3, and an opening 12c provided between the pinch 12a and the pressure plate 12b.

Additionally, a spring 13 is disposed at the left side end of this lid 12, and this spring 13 biases the lid 12 toward the right side at all times. Thus, the opening 12c of the lid 12, and the opening 10 of the main body case 3 are kept in a disagreed state during carrying or measuring the blood pressure, as shown in FIG. 4.

Therefore, a liquidified aromatic antihypertensive agent stored in the antihypertensive agent accommodating section 9 never flows out from the opening 10 of the main body case 3 during carrying or measuring the blood pressure.

When the operator who observes the systolic blood pressure and diastolic blood pressure values displayed on the display unit 8 deems that the blood pressure is high after the measurement of the blood pressure, the operator manipulates the switch 14 positioned on the lower left side of the main body case 3 shown in FIG. 3.

Thus, the control element 3b shown in FIG. 5 switches an air duct switching means 15 shown in FIG. 4 from a draft air duct 5a toward the pressurization bag 4, to a draft air duct 5b toward the antihypertensive agent storage section 9. This switching results in transfer of the air blasting from the blower (air blasting pump) 6 through the air duct switching means 15 and the draft air duct 5b, into the antihypertensive agent storage unit 9.

The air blasting from the blower (air blasting pump) 6 transferred into the antihypertensive agent storage unit 9 flows into the antihypertensive agent storage unit 9 while pushing the pressure plate 12b at the right side end of the lid 12 toward the left side in FIG. 4.

Then, when the movement of this lid 12 to the left side in FIG. 4 results in agreement of the opening 12c of the lid 12 with the opening 10 of the main body case 3, the aromatic antihypertensive agent (i.e., the antihypertensive agent comprising citral and linalool with a mass ratio of citral to linalool being not less than 10:1 and not greater than 40:1) components in the antihypertensive agent storage section 9 flows out by being conveyed with the air blasting through the opening 10 of the main body case 3. Consequently, the operator of the measurement of the blood pressure smells these components. In this step, since the aromatic antihypertensive agent components flow out from the opening 10 while being conveyed with the air blasting, the user may not move the nostril toward the opening 10 beyond necessity, making the use of the device very convenient.

After smelling of this aromatic antihypertensive agent components, manipulating the switch 14 again stops the operation of the blower (air blasting pump) 6 by the control element 3b, and the air duct switching means 15 is also switched to the draft air duct 5 toward the pressurization bag 4. Therefore, pushing of the lid 12 to the right side occurs with the spring 13, leading to the state shown in FIG. 4. As a result, the smelling behavior of the aromatic antihypertensive agent components is terminated.

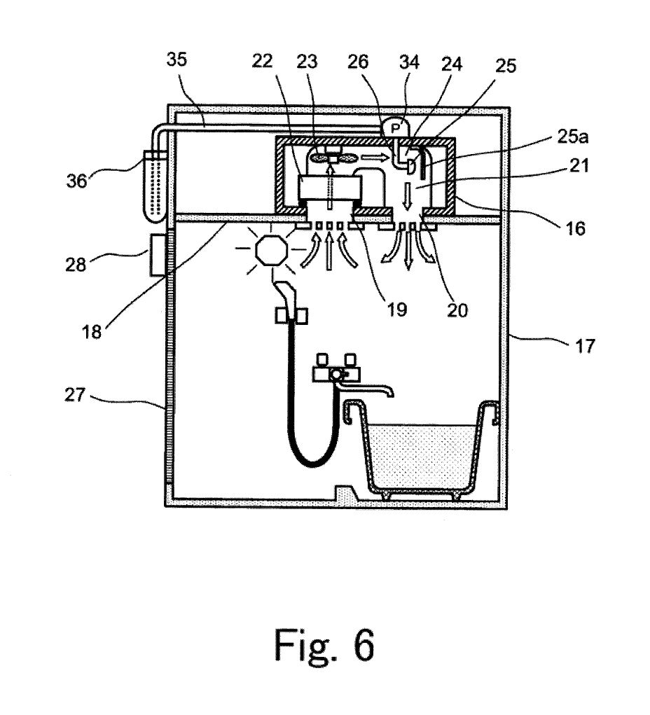

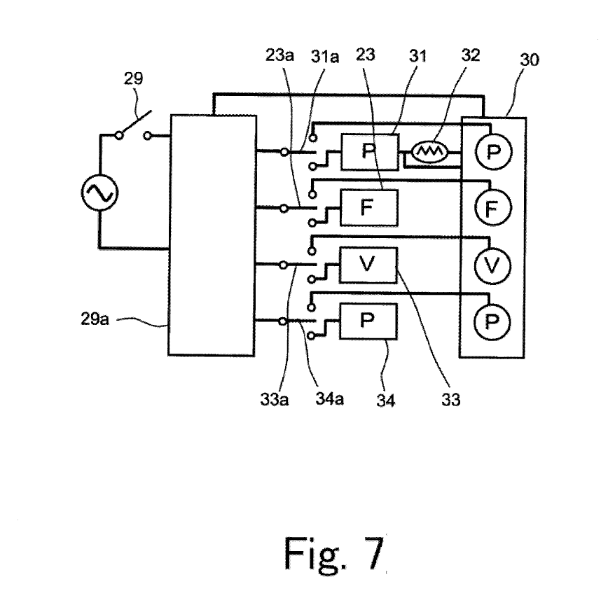

FIG. 6 and FIG. 7 show a sauna apparatus which can be utilized as one exemplary electronic device for inhalation of the antihypertensive agent of the present invention through the nostril of a mammal.

In FIG. 6, a numeral 16 indicates a main body case of a sauna apparatus, and this main body case 16 is installed on the ceiling 18 of a bathroom 17.

The main body case 16 has an air inlet port 19 for inflowing the air from the bathroom 17, and an exhaust vent 20 for exhausting the air into the bathroom 17. In this main body case 16, a warm water type heater 22, a blower 23, and a humidifier 24 are arranged sequentially in a draft air duct 21 connecting the air inlet port 19 and the exhaust vent 20.

The humidifier 24 has a nozzle 25 as a means for generating fine liquid droplets, and this nozzle 25 is connected to a water supply pipe 26 as a water supply means. A portion of this water supply pipe 26 is in contact with a heater 22 although not shown in the figure, thermal conduction from the heater 22 results in heating of water in the water supply pipe 26. Accordingly, warm water is sprayed from the nozzle 25.

More specifically, for enjoying sauna in the bathroom 17, a switch 29 shown in FIG. 7 provided in the control board 28 on the outside face of a door 27 of the bathroom 17 is first closed to permit conduction in an electric source circuit 29a.

Thus, a controller 30 shown in FIG. 7 closes a switch 31a to drive a pump 31, and thus warm water from a water heater (not shown in the figure) flows into the heater 22.

When a temperature sensor 32 detects elevation of the temperature to a predetermined temperature of the heater 22 by warm water inflow, the switch 23a is closed to drive the blower 23.

When the blower 23 is driven, the air in the bathroom 17 is first fed into the main body case 16 from the air inlet port 19 as shown in FIG. 6, next heated by the heater 22, and thereafter emitted from the exhaust vent 20 into the bathroom 17. Accordingly, heating in the bathroom 17 is permitted.

When the heating in the bathroom 17 is initiated in this manner under such circumstances, warm water is supplied through the water supply pipe 26 to the nozzle 25 at a rate of 60 cc per min. Accordingly, the warm water is flushed from the nozzle 25 toward the impingement plate 25a.

The impingement of warm water onto the impingement plate 25a makes almost the warm water fine at the impingement plate 25a portion, which is then heated by the heater 22 and conveyed to the warm air that is moving toward the exhaust vent 20, and then supplied to a bathroom 17 while being evaporated. Thus, preparation for enjoying sauna in the bathroom 17 is completed.

For enjoying sauna in the bathroom 17, in a state in which heating in the bathroom 17 and warm water spraying from the nozzle 25 are kept on for no shorter than 5 min, the user will enter into the bathroom 17.

During staying in the bathroom, the user receives continuously heating in the bathroom 17 and warm water spraying from the nozzle 25.

The foregoing explanation has been made for briefly explaining fundamental configuration and operation of the sauna apparatus, and hereafter, specific features of the present sauna apparatus will follow.

The features of the present sauna apparatus are pertinent to linkage between the water supply pipe 26 shown in FIG. 6 and an antihypertensive agent storage unit 36 through a valve 33 shown in FIG. 7, and also through a pump 34 and a pipe 35 shown in FIG. 6 and FIG. 7.

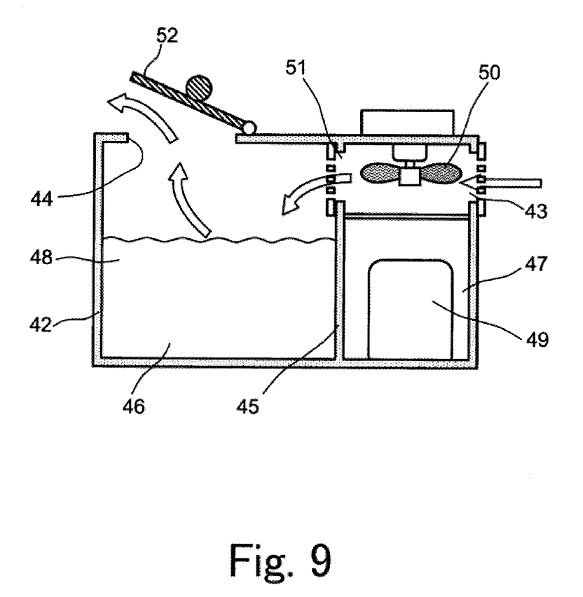

The antihypertensive agent storage unit 36 is preferably provided outside the main body case 16 for ease in filling the aromatic antihypertensive agent, and in the present sauna apparatus, it is provided detachably outside the door 27 of the bathroom 17 as shown in FIG. 9.

The antihypertensive agent storage unit 36 is filled with a liquidified aromatic antihypertensive agent.

According to the present sauna apparatus, for example, the valve 33 is intermittently opened and the pump 34 is driven while operating the sauna.

Specifically, during the operation of the sauna (i.e., the state in which heating in the bathroom 17, and warm water spraying of the nozzle 25 have been continuously carried out), switches 33a and 34a of the controller 30 are closed only for 30 sec, once every five minutes, whereby the pump 34 is driven to open the valve 33.

Thus, the liquidified antihypertensive agent filled in the antihypertensive agent storage unit 36 is flowed into the water supply pipe 26 through the pipe 35.

Warm water flows through the water supply pipe 26 to the nozzle 25 at a rate of 60 cc per min, and thus the aromatic antihypertensive agent is mixed with the warm water and flushed from the nozzle 25 toward the impingement plate 25a.

The impingement onto the impingement plate 25a makes most of the warm water and the antihypertensive agent fine at the impingement plate 25a portion, which are then heated by the heater 22 and conveyed to the warm air that is moving toward the exhaust vent 20, and then supplied to a bathroom 17 while being evaporated.

Therefore, the user can smell the aromatic antihypertensive agent components concurrently with entering the bathroom 17 without need of moving the nostril toward the exhaust vent 20, whereby the system is made very convenient.

In the present sauna apparatus, for the purpose of placing importance on functions of the sauna apparatus anyway, the preset amount of warm water sprayed from the nozzle 25 is much more than that of the aromatic antihypertensive agent sprayed.

Conversely, the present amount of the aromatic antihypertensive agent mixed in the warm water is less than that of the warm water. However, the bathroom 17 can be sufficiently filled with the aromatic antihypertensive agent components (i.e., an antihypertensive agent comprising citral and linalool, having a mass ratio of citral to linalool being not less than 10:1 and not greater than 40:1) even with such settings.

Although the present sauna apparatus is configured such that the liquidified antihypertensive agent filled in the antihypertensive agent storage unit 36 is flowed into the water supply pipe 26 through the pipe 35 in the state unheated, a configuration in which a portion of the pipe 35 is brought into contact with the heater 22 to allow the antihypertensive agent in the pipe 35 to heat by way of thermal conduction from the heater 22, and the heated antihypertensive agent is flowed into the water supply pipe 26 is also acceptable.

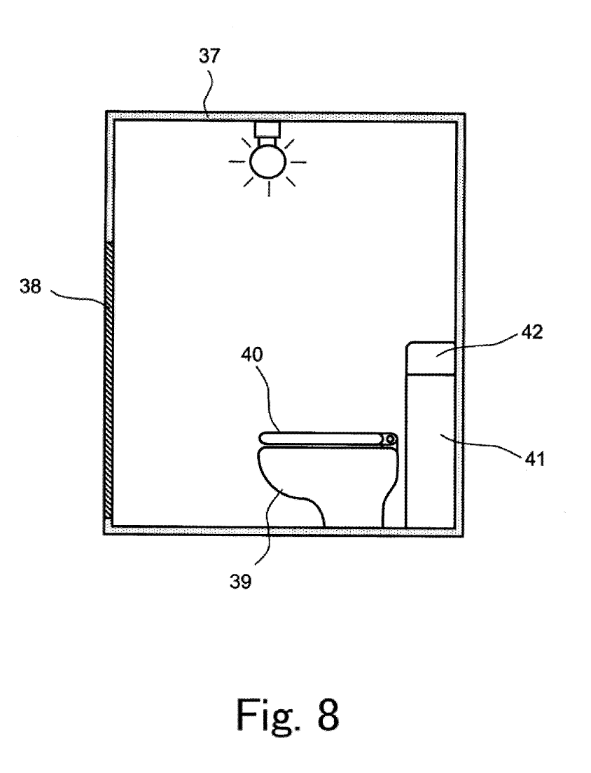

FIG. 8 to FIG. 10 show a warming toilet seat which can be utilized as one exemplary electronic device for inhalation of the antihypertensive agent of the present invention through the nostril of a mammal.

In FIG. 8, a numeral 37 indicates a toilet booth, provided with a door 38 at the entrance which is openable and closable, and a toilet bowl 39 is provided inside.

In addition, a warming toilet seat 40 is provided at the upper opening of the toilet bowl 39. Furthermore, a waste water tank 41 is provided backside of the toilet bowl 39, and a main body case 42 containing the aromatic antihypertensive agent is provided on the upper side of the waste water tank 41.

The main body case 42 has a box-like shape as shown in FIG. 9, and an air inlet port 43 and an exhaust vent 44 are provided on the upper side.

A partition plate 45 separates the main body case 42 into left and right compartments as shown in FIG. 9, with the compartment on the left side forming an antihypertensive agent storage unit 46, and the right side forming a controller cabinet 47.

The antihypertensive agent storage unit 46 on the left side stores a carrier 48 such as sponge or cotton impregnated with the liquidified aromatic antihypertensive agent.

The controller cabinet 47 on the right side includes a controller 49 and a blower 50.

Accordingly, driving of the blower 50 first leads to aspiration of the air in the toilet booth 37 from the air inlet port 43 of the main body case 42. Subsequently, this air flows into the antihypertensive agent accommodating section 46 through the opening 51 upside of the partition plate 45.

Since the antihypertensive agent storage unit 46 has the carrier 48 impregnated with the liquidified antihypertensive agent as described above placed therein, the aromatic antihypertensive agent components (i.e., the antihypertensive agent comprising citral and linalool, and having a mass ratio of citral to linalool of not less than 10:1 and not greater than 40:1) are conveyed while being evaporated when the air passes the upper surface of the carrier 48, end emitted from the exhaust vent 44 into the toilet booth 37.

Although the exhaust vent 44 has a lid 52 which is openable and closable, this lid 52 is designed so as to be opened by the air blasting pressure upon driving of the blower 50 as shown in FIG. 7.

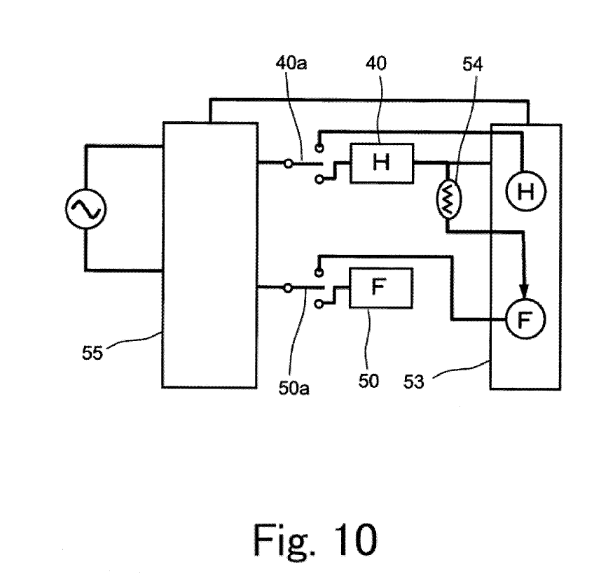

FIG. 10 shows a control circuit, in which a controller 53 is designed to execute the control of electrification of the warming toilet seat 40.

More specifically, in order to maintain the warming toilet seat 40 to have a preset temperature, detection of the temperature with a sensor 54 results in turning on/off of the switch 40a.

According to one aspect in the warming toilet seat of the present embodiment, the warming toilet seat 40 is more frequently electrified as the ambient temperature is lower, particularly in low-temperature seasons such as winter in Japan, and thus the present warming toilet seat is designed to drive the blower 50 more frequently as the ambient temperature becomes even lower.

More specifically in connection with this point, since it is necessary to exert greater control over the blood pressure elevation during using a toilet booth 37 as the ambient temperature is lower, the blower 50 for the warming toilet seat is driven more frequently as the ambient temperature becomes lower as described above to permit emission of the aromatic antihypertensive agent components into the toilet booth 37.

Furthermore, since the toilet booth 37 is used suddenly and irregularly depending on the user's circumstances, coordination of the electrification of the blower 50 to electrification timing of the warming toilet seat 40 to allow the aromatic antihypertensive agent components to be automatically conveyed and emitted into the toilet booth 37 like the present warming toilet seat will result in superior user-friendliness to meet the utilization mode.

When a warm water showering apparatus (not shown in the figure) for use in sitting the warming toilet seat 40 is employed, a configuration in which electrification of the heater (not shown in the figure) for heating water for the showering is coordinated to driving of the blower 50 is also acceptable. It should be noted that a numeral 55 in FIG. 12 indicates an electric source circuit.

The aromatic antihypertensive agent used in each electronic device illustrated in FIG. 2 to FIG. 10 is a mixture of citral and linalool, and a liquidified mixture prepared by dilution with a mineral oil is preferably used.

In addition, since lowering of the blood pressure was observed with a composition of the mixture having a mass ratio of citral to linalool being 10:1, 20:1 or 40:1, the proper mass ratio may be selected from among these. However, the mass ratio is most preferably approximately 20:1 because the greatest effect was exhibited.

From the foregoing description, many modifications and other embodiments of the present invention are apparent to persons skilled in the art. Accordingly, the foregoing description should be construed merely as an illustrative example, which was provided for the purpose of teaching best modes for carrying out the present invention to persons skilled in the art. Details of the construction and/or function of the present invention can be substantially altered without departing from the spirit thereof.

### INDUSTRIAL APPLICABILITY

The aromatic composition according to the present invention has an efficacious antihypertensive action, and is useful as an antihypertensive agent for hypertensive or susceptible subjects (patients).

1-4. (canceled)

5. A method for lowering blood pressure in a mammal, comprising,
allowing the mammal to inhale an aromatic composition comprising citral and linalool through a nostril of the mammal, wherein
the aromatic composition has a mass ratio of the citral to the linalool of not less than 10 and not greater than 40.

6. The method according to claim 5, wherein the mass ratio is 20.
